(19) United States
(12) Patent Application Publication
(10) Pub. No.: US 2005/0014198 A1
(43) Pub. Date: Jan. 20, 2005

(54) Assays and kits for detecting and monitoring heart disease

(76) Inventor: Leong Ng, Leicester (GB)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/835,084

(22) Filed: Apr. 29, 2004

Related U.S. Application Data
(63) Continuation-in-part of application No. 10/618,567, filed on Jul. 11, 2003.

(30) Foreign Application Priority Data
Jan. 19, 2004 (GB) 0401085.6
Jul. 17, 2002 (GB) 0216505.8
Jul. 16, 2002 (GB) 0216500.9
Jul. 11, 2002 (GB) 0216191.7

(51) Int. Cl. G01N 33/53

(52) U.S. Cl. 435/7.1

(57) ABSTRACT

The invention provides methods and a kit for detecting an increased risk of a heart condition in a subject by detecting an increased level of urotensin II in the subject's bodily fluid.

### RELATED APPLICATIONS

This application is a continuation in part of Ser. No. 10/618,567, filed Jul. 11, 2003.

### BACKGROUND OF THE INVENTION

Heart disease is a major health burden in developed countries, and its main aetiology is atherosclerosis. Accumulation of lipid, especially oxidized or modified LDL, together with macrophages and other cells, leads to plaque growth and instability. Rupture of these plaques leads to thrombosis and a resulting occlusion of the coronary arterial lumen presents as an acute coronary syndrome (ACS). Acute coronary syndromes include myocardial infarction (MI) and unstable angina (UA).

Acute coronary syndromes are major health problems throughout the world, but currently there are limited means for effective diagnosis and risk stratification of patients. Biochemical techniques involving measurement of various blood analytes are currently utilized to achieve these objectives. For example, Brain natriuretic peptide (BNP) or its N-terminal precursor, N-terminal proBrain natriuretic peptide (N-BNP), are secreted during acute cardiac ischaemia, and may provide prognostic information about the patient (Omland, et al., Circulation 2002, 106: 2913-8; Richards, et al., Circulation 2003, 107: 2786-92).

To date, most diagnostic procedures for acute coronary syndromes and heart failure generally assess the extent of cardiac tissue damage after clinical signs have appeared. These methods of identifying and confirming heart failure require more time than is often available in emergency situations where rapid evaluation is critical for effective patient treatment and survival. In an emergency medical facility, electrocardiography (ECG) monitoring of suspected patients is the most rapid diagnostic method for detecting acute myocardial infarction (Mair et al, Coron Artery Dis 1995, 6: 539-45; Mairetal., Chest 1995, 108: 1502-9).

Electrocardiography and currently available diagnostic blood tests are generally not effective for early detection of heart disease that precedes the damage associated with heart attacks, because these tests detect infarction-associated tissue damage. Currently, the only diagnostic for chronic underlying coronary artery disease is ECG monitoring during exercise stress (e.g., treadmill exercise). Further, ECG is generally used to confirm the clinical symptoms of angina (chest pain). Such stress testing is usually given after the patient has experienced symptoms and sought treatment (e.g., at an emergency medical facility). Although stress testing is sometimes used to screen asymptomatic patients, testing is costly, time-consuming, and generally not amenable to routine screening of large numbers of patients. Furthermore, exercise stress test evaluations result in about 15% false negatives.

Diagnostic tests have been developed that use cardiac proteins to determine whether or not the source of the patient's chest pain is cardiac, and if so, whether the patient has suffered a myocardial infarct or is suffering from unstable angina (see, e.g., U.S. Pat. Nos. 5,290,678; 5,604,105; 5,710,008). Other diagnostic tests use non-polypeptidic cardiac markers for the early detection of heart disease (see U.S. Pat. No, 6,534,322).

Accordingly, there remains a need for a better non-invasive, more sensitive, and highly reliable point-of-care ‘bedside test’ for the early detection of heart disease and acute coronary syndromes.

### SUMMARY OF THE INVENTION

The disclosed invention is based on the finding that the levels of urotensin II in bodily fluids are raised in patients at increased risk for heart failure and acute coronary syndromes (ACS). The invention as disclosed herein provides patients with sensitive and reliable assays and kits to detect an increased risk of heart conditions.

In a first aspect, the invention provides methods for determining an increased risk of a heart condition, including heart failure and/or acute coronary syndromes, in a subject or for identifying subjects who would benefit from revascularization therapy by quantitating an increased level of urotensin II, in a bodily fluid of the subject, whereby an elevated level of urotensin II relative to the normal level is indicative of an increased risk of a heart condition. The subject may be a mammalian subject, and preferably, is a human. Bodily fluids include, but are not limited to, plasma, interstitial fluid, urine, whole blood, serum, or saliva.

In a preferred embodiment, the level of at least one further marker indicative of a heart condition, such as N-terminal pro-brain natriuretic peptide (N-BNP), brain natriuretic peptide (BNP), atrial natriuretic peptide (ANP), C-type natriuretic peptide (CNP), Troponin T, creatine kinase MB isoform (CKMB), or myoglobin, is measured, wherein an elevated level of the further marker relative to the normal is indicative of an increased risk of a heart condition.

The level of urotensin II alone or in combination with another marker may be determined by use of an immunoassay.

In a second aspect, the invention provides a kit for quantitating the relative amount of urotensin II in a bodily fluid incorporating the methods of the first aspect. Such a kit may comprise one or more reagents for quantitating the level of urotensin in a bodily fluid. The one or more reagents may comprise an antibody that is immunospecific for urotensin.

Other features and advantages will be appreciated based on the following Detailed Description and Claims.

### BRIEF DESCRIPTION OF THE DRAWINGS

FIG. 1 contains a table which shows patient characteristics (Medians [ranges] are reported and P values were computed using the Kruskal-Wallis or Mann Whitney tests (comparing normal and heart failure patients)).

FIGS. 2a and 2b are graphs showing plasma N-terminal pro brain natriuretic peptide and plasma urotensin, respectively, according to New York Heart Association class.

FIGS. 3a and 3b are Receiver Operating Characteristic (ROC) curves for N-terminal pro brain natriuretic peptide (N-BNP) and urotensin (UTN), respectively, in the diagnosis of heart failure. The arrows indicate the distance from the top left corner and the curve.

FIG. 4 is a ROC curve prognostic index for combination of N-terminal pro brain natriuretic peptide (N-BNP) and urotensin (UTN) in the diagnosis of heart failure. The arrow indicates the distance from the top left corner and the curve.

FIGS. 5a and 5b show plasma levels of urotensin and N-BNP, respectively, in patients with Myocardial Infarction (MI) or Unstable Angina (UA) compared to normal subjects not suffering from MI or UA.

FIGS. 6a and 6b show Receiver Operating Characteristic (ROC) curves for the diagnosis of Acute Coronary Syndromes using plasma urotensin and plasma N-BNP, respectively.

FIGS. 7a and 7b show plasma levels of urotensin and N-BNP, respectively, in patients with ACS who either had an uncomplicated clinical course or were subsequently rehospitalized with a further event (MI or UA).

FIGS. 8a and 8b show plasma levels of urotensin and BNP, respectively, in patients with stable angina, undergoing coronary angiography and either having a balloon angioplasty or no intervention to their coronary arteries. Levels of both markers peak at 2 hours after the balloon angioplasty.

### DETAILED DESCRIPTION OF THE INVENTION

1. Definitions

For convenience, before further description of the disclosed invention, certain terms employed in the specification, examples, and appended claims are provided here.

The singular forms “a”, “an”, and “the” include plural references unless the context clearly dictates otherwise.

The terms “acute coronary syndrome” or “ACS,” as used herein, refer to myocardial infarction (MI) and unstable angina, but may also include new-onset angina and sudden cardiac death. ACS may result from atherosclerosis or coronary artery disease, wherein the instability and then rupture of atherosclerotic plaques may lead to thrombotic occlusion of coronary arteries. ACS may also develop as a result of a heart condition.

“ANP” refers to atrial natriuretic peptide, the first described peptide in a family of hormones which regulate body fluid homeostasis (Brenner et al., Physiol. Rev. 1990; 70: 665).

The term “antibody,” as used herein, refers to binding molecules including immunoglobulin molecules and immunologically active portions of immunoglobulin molecules, i.e., molecules that contain an antigen binding site that specifically bind an antigen. The immunoglobulin molecules useful in the invention can be of any class (e.g., IgG, IgE, IgM, IgD, and IgA) or subclass of immunoglobulin molecule. Antibodies include, but are not limited to, polyclonal, monoclonal, bispecific, partially or fully humanized, chimeric antibodies, single chain antibodies, Fab fragments (F(ab′) and F(ab′)₂), fragments produced by a Fab expression library, anti-idiotypic (anti-Id) antibodies, and epitope-binding fragments of any of the above. An antibody, or generally any molecule, “binds specifically” to an antigen (or other molecule) if the antibody binds preferentially to the antigen, and, e.g., has less than about 30%, preferably 20%, 10%, or 1% cross-reactivity with another molecule. Portions of antibodies include Fv and Fv′ fragments.

The term “bodily fluid,” as used herein, includes all fluids that can be obtained from a mammalian body, including, for example, whole blood, plasma, urine, interstitial fluid, lymph, gastric juices, bile, serum, saliva, sweat, and spinal and brain fluids. Furthermore, a bodily fluid may be either processed (e.g., serum) or present in its natural form.

As used herein, the term “brain natriuretic peptide” includes a native brain natriuretic peptide (BNP), N-terminal pro-BNP (N-BNP), pro-BNP (propeptide form), and BNP-signal peptide (BNP-SP) as well as portions thereof.

“CNP” refers to C-type natriuretic peptide (Stingo et al., Am. J Physiol., 1992; 263: H1318-21).

“Comprise” and “comprising” are used in the inclusive, open sense, meaning that additional elements may be included.

The term “diagnosis,” as used herein, refers to the identification of a disease in a subject or the subject's susceptibility to develop the disease.

The term “heart condition,” as used herein, refers to a wide range of abnormalities and/or diseases of the heart, coronary vasculature, or blood vessels surrounding the heart including underlying conditions, such as, ischemia (including, for example, atherosclerosis (coronary artery disease), embolism, congenital heart defects, anemia, lung disease, and abnormal stimulation (e.g., sympathomimetic abuse)), hypertension (including, for example, systemic hypertension (e.g., primary and secondary) and pulmonary hypertension (e.g., chronic obstructive pulmonary disease, restrictive lung disease, pulmonary embolism, and morbid obesity)), valvular disease (including, for example, mitral valve disease, aortic valve disease, tricuspid valve disease and pulmonary valve disease), heart muscle disease (including, for example, ischemic cardiomyopathy, dilated cardiomyopathy, hypertensive cardiomyopathy, hypertrophic cardiomyopathy, restrictive cardiomyopathy, and specific heart muscle disease resulting from cardiac infection (i.e. bacterial or viral infection), toxins, metabolites, neuromuscular disease, storage disorders, infiltration disorders, and immunologic disorders), pericardial disease, rheumatoid heart disease, neoplastic heart disease (including, for example, primary cardiac tumors), coronary vasospasm (including, for example, drug induced vasospasm), cardiac trauma, and genetic or hereditary predisposition that may manifest as angina (including, for example, stable angina, unstable angina, mixed angina, and Prinzmetal's variant angina), myocardial infarction, chronic ischemic heart disease, and sudden cardiac death.

The term “heart failure,” as used herein, refers to the inability of the heart to pump blood with normal efficiency. Heart failure may result from cardiac or extra-cardiac causes. Cardiac causes can result from heart disease. Extra-cardiac causes can result from metabolic disorders (including, for example, hyperthyroidism, Paget's disorder, vitamin deficiency (such as beriberi), anemia, arteriovenous fistula, and infection (including, for example, bacterial, viral, or toxin).

The term “immunoassay,” as used herein, refers to an assay that utilizes an antibody to specifically bind to a protein.

The term “marker” or “marker protein,” as used herein, refers to the amount of polypeptide or peptide in bodily fluid that is indicative of a disease and can be quantitated.

As used herein, the term “natriuretic peptide” includes a native ANP, BNP, or CNP, portions of, variants of, or chimeras thereof.

The term “NYHA classification” refers to the New York Heart Association (NYHA) classification. This is a four-stage classification where:

Class 1 refers to patients that exhibit symptoms only at exertion levels;

Class 2 refers to patients that exhibit symptoms with ordinary exertion;

Class 3 refers to patients that exhibit symptoms with minimal exertion;

Class 4 refers to patients exhibit symptoms at rest.

The terms “quantify” and “quantitate,” as used herein, refer to the process of measuring the amount of a marker (e.g., in terms of its concentration, mass, moles, or volume in a sample).

A “reagent” refers to a substance or molecule that binds or interacts with a polypeptide or peptide.

A “subject” refers to a human or a non-human animal.

The terms “urotensin” or “UTN,” as used herein, refer to urotensin II (see GenBank Accession Numbers NM_{—}021995 and NM_{—}006786) and fragments and variants thereof (e.g., allelic variants). Urotensin is derived from a prohormone precursor, pro-urotensin (GenBank Accession Number 095399), which is processed to mature urotensin and an N-terminal peptide. The term “urotensin,” as used herein, further includes pro-urotensin, mature urotensin, its N-terminal derived peptide, its signal peptide, and a C-terminal peptide (Glu-Thr-Pro-Asp-Cys-Phe-Trp-Lys-Tyr-Cys-Val (disulphide bond between Cys⁵ and Cys¹⁰) (Seq ID NO: 1)) as well as fragments thereof. The term urotensin also refers to urotensin related peptide (URP) (Ala-Cys-Phe-Trp-Lys-Tyr-Cys-Val (disulphide bond between Cys² and Cys⁷) (Seq ID NO: 2)) as well as the proform of urotensin related peptide (GenBank Accession Number NM_{—}198152).

2. General

UTN is known to be a marker for heart failure (Douglas, et al., Lancet 2002, 359: 1990-1997). We now show that UTN is present in plasma at increased levels in subjects with heart failure and/or acute coronary syndromes (ACS) and in subjects undergoing cardiac ischaemia (e.g. resulting from balloon angioplasty). These unexpected findings indicate that UTN may also be a sensitive and early marker for diagnosing heart conditions and/or myocardial damage incurred during angioplasty or as a result of ACS.

3. Methods of Diagnosing Heart Conditions

Based on the above, the invention features methods for diagnosing heart conditions in a subject including heart failure or acute coronary syndromes by quantitating the level of urotensin in a subject's bodily fluid. The measured amount of urotensin (and, where measured, other marker(s) indicative of a heart condition) may be compared with a normal amount (i.e. the amount of urotensin (or other marker(s)) present in a subject without a heart condition). The reference or normal levels of urotensin indicative of the absence of a heart condition may range from about 3-10 fmol/ml. A subject may be matched for age and/or gender.

The level of urotensin II may be measured from a bodily fluid, such as, blood, plasma, urine, lymph, gastric juices, bile, serum, saliva, sweat, and spinal and brain fluids. Furthermore, the bodily fluids may be either processed (e.g., serum) or unprocessed. Methods of obtaining a bodily fluid from a subject are known to those skilled in the art. Levels of urotensin that are indicative of an increased risk of a heart condition are greater than 10 fmol/ml, such as, for example, 10-15 fmol/ml, 10-20 fmol/ml, 10-25 fmol/ml, 10-30 fmol/ml or more.

In the invention, the level of urotensin may be quantitated in a bodily fluid using an antibody or portion thereof that binds specifically to urotensin. Specific antibodies may be directed against any portion of urotensin, including the signal peptide. Antigenic fragments within urotensin may be identified by methods well-known in the art. Fragments containing antigenic sequences may be selected on the basis of generally accepted criteria of potential antigenicity and/or exposure. Such criteria include the hydrophilicity and relative antigenic index, as determined by surface exposure analysis of proteins. The determination of appropriate criteria is well-known to one of skill in the art, and has been described, for example, by Hopp et al., Proc Natl Acad Sci USA 1981; 78: 3824-8; Kyte et al., J Mol Biol 1982; 157: 105-32; Emini, J Virol 1985; 55: 836-9; Jameson et al., CA BIOS 1988; 4: 181-6; and Karplus et al., Naturwissenschaften 1985; 72: 212-3. Amino acid domains predicted by these criteria to be surface exposed may be selected preferentially over domains predicted to be more hydrophobic.

Portions of urotensin determined to be antigenic may be chemically synthesized by methods known in the art from individual amino acids. Suitable methods for synthesizing protein fragments are described by Stuart and Young in “Solid Phase Peptide Synthesis,” Second Edition, Pierce Chemical Company (1984).

If a portion of urotensin defines an epitope, but is too short to be antigenic, it may be conjugated to a carrier molecule in order to produce antibodies. Some suitable carrier molecules include keyhole limpet hemocyanin, Ig sequences, TrpE, and human or bovine serum albumen. Conjugation may be carried out by methods known in the art. One such method is to combine a cysteine residue of the fragments with a cysteine residue on the carrier molecule.

Polyclonal and monoclonal antibodies may be produced by methods known in the art. Monoclonal antibodies may be produced by hybridomas prepared using known procedures including the immunological method described by Kohler and Milstein, Nature 1975; 256: 495-7; and Campbell in “Monoclonal Antibody Technology, The Production and Characterization of Rodent and Human Hybridomas” in Burdon et al., Eds. Laboratory Techniques in Biochemistry and Molecular Biology, Volume 13, Elsevier Science Publishers, Amsterdam (1985); as well as by the recombinant DNA method described by Huse et al., Science 1989: 246: 1275-81.

Other embodiments include functional equivalents of antibodies, and include, for example, chimerized, humanized, and single chain antibodies as well as fragments thereof. Methods of producing functional equivalents are disclosed in PCT Application WO 93/21319; European Patent Application No. 239,400; PCT Application WO 89/09622; European Patent Application 338,745; and European Patent Application EP 332,424.

Antibodies binding to urotensin may also be obtained commercially. Examples of commercially available antibodies binding to urotensin include anti-urotensin (Phoenix Pharmaceuticals), rabbit anti-urotensin (Biodesign International), and rabbit anti-human urotensin (Immundiagnostik).

In further embodiments, a second marker indicative of a heart condition may be detected and/or quantitated in combination with urotensin. These markers may be one or more of brain natriuretic peptide, atrial natriuretic peptide, C-type natriuretic peptide, troponin, creatine kinase MB isoform (CKMB), and myoglobin, as well as fragments and precursors thereof.

For example, myocardial stretch, myocardial tension, and myocardial injury trigger increased production of pro-brain natriuretic peptide (proBNP) from cardiac myocytes in the left ventricle. proBNP is the intact precursor to the two circulating forms, BNP (the active peptide) and N-terminal proBNP (N-BNP, the inactive peptide). The level of N-BNP and/or BNP may be measured. Other natriuretic peptides, such as atrial natriuretic peptide (ANP), N-terminal proANP (N-ANP) (Hall, Eur J Heart Fail, 2001, 3:395-397), and C-type natriuretic peptide (CNP) (Suga et al., J Clin, Invest., 1992, 90:1145; Stingo et al., Am. J. Physiol., 1992, 262:H308; Stingo et al., Am. J Physiol., 1992, 263:H1318; Koller et al., Science, 1991, 252:120) have also been described as markers of cardiac disease and may be measured.

Troponins are also useful markers of myocardial damage (James et al, Circulation, 2003, 108:275-81). Similarly, creatine kinase MB isoform (CKMB) and myoglobin are currently used for the diagnosis of myocardial infarction, as the levels of each are elevated more rapidly than the troponins.

Other secondary markers that could be used to diagnose heart conditions may include non-polypeptidic cardiac markers such as sphingolipid, sphingosine, sphingosine-1-phosphate, dihydrosphingosine and sphingosylphosphorylcholine (see U.S. Pat No. 6,534,322).

The level of a second marker may be quantitated and compared with a normal level of the second marker, which is indicative of the absence of a heart condition. The normal level may be the amount of a second marker from one or more mammalian subjects free from a heart condition, or with a previously determined reference range for the second marker in mammalian subjects free from a heart condition. Where measured, the normal value of N-BNP that is indicative of the absence of a heart condition may range from about 10-50 fmol/ml. Levels of N-BNP that are indicative of an increased risk of a heart condition may range from about 100-300 fmol/ml, 300-600 fmol/ml, 600-1200 fmol/ml, 1200-1800 fmol/ml, 1800-2400 fmol/ml, 2400-3000 fmol/ml, 3000-3600 fmol/ml or more.

The normal level of myoglobin that is indicative of the absence of ACS may vary according to the device used to measure the level of myoglobin (Le Moigne et al Clin Biochem., 2002, 35(4):255-62). For example, the normal level may be up to about 92 μg/L for men and about 76 μg/L for women if measured by Olympus, up to about 46 μg/L if measured by Vidas or up to about 70 μg/L if measured by Immulite Turbo. Another study suggests that the normal level may be up to about 65 μg/L for men and about 55 μg/L for women (Penttila et al, Clin Biochem. 2002, 35(8):647-53). Levels of myoglobin which exceed the above normal levels may be indicative of ACS.

The levels of troponins, including Troponin T and Troponin I, may also be measured. For Troponin T, the cut off for acute myocardial infarction may be about 0.05 μg/L (Collinson et al, Heart. 2003, 89(3):280-6). James et al. (J Am Coll Cardiol. 2003, 41(6):916-24) also establishes Troponin T levels as an indicator of myocardial infarction (MI). For Troponin I, the level for diagnosis of ACS (including MI) may be about 0.6 μg/L or above (Apple et al, Clin Chem. 2000, 46(4):572-4).

For CKMB, the level for diagnosis of ACS (including MI) may be about 5 μg/L or more (Falahati et al. Am Heart J. 1999, 137(2):332-7). Values below these respective levels may indicate the absence of ACS. When measuring the levels of a second marker(s), corrections for age and gender may be necessary in order to improve the accuracy of diagnosis.

Examples of commercially-available antibodies binding to BNP are rabbit anti-human BNP polyclonal antibody (Biodesign International), rabbit anti-BNP amino acids 1-20 polyclonal antibody (Biodesign International), anti-human BNP monoclonal antibody (Immundiagnostik), and rabbit anti-human BNP amino acids 1-10 polyclonal antibody (Immundiagnostik). Examples of commercially available antibodies binding to ANP are mouse anti-human ANP monoclonal antibody (Biodesign International), rabbit anti-human ANP monoclonal antibody (Biodesign International), mouse anti-human ANP monoclonal antibody (Chemicon), rabbit anti-human ANP amino acids 95-103 antibody (Immundiagnostik), rabbit anti-human ANP amino acids 99-126 antibody (Immundiagnostik), sheep anti-human ANP amino acids 99-126 antibody (Immundiagnostik), mouse anti-human ANP amino acids 99-126 monoclonal antibody (Immundiagnostik) and rabbit anti-human a-ANP polyclonal antibody (United States Biological). Examples of commercially available antibodies binding to CNP include rabbit anti-C-Type Natriuretic Peptide-22 (Phoenix Pharmaceuticals). Antibodies binding to troponins, creatine kinase MB isoform and myoglobin can be obtained from Research Diagnostics, Inc., for example.

Depending on the assay used to diagnose a heart condition (see below), the antibodies specific to the markers of a heart condition may further comprise a label, e.g., a fluorescent moiety, an enzyme, a magnetic label, a latex or gold particle, an electrochemically active species, etc. In embodiments where the label is attached to the antibody, the antibody is said to be “directly labeled.” An antibody can also be “indirectly labeled,” i.e., the label is attached to the antibody through one or more other molecules, e.g., biotin-streptavidin. Alternatively, the antibody is not labeled, but is later contacted with a binding agent after the antibody is bound to a specific marker. For example, there may be a “primary antibody” and a second antibody or “secondary antibody” that binds to the Fc portion of the first antibody. Labels may be linked, preferably covalently, to antibodies according to methods known in the art. In an immunoassay, the presence or amount of analyte (or marker) present is determined by detection of the presence or concentration of the label.

Further depending on the assay used to diagnose a heart condition, antibodies may be linked to a solid surface. The solid surface can be selected from a variety of those known in the art including plastic tubes, beads, microtiter plates, latex particles, gold particles, magnetic particles, cellulose beads, agarose beads, paper, dipsticks, and the like. Methods for direct chemical coupling of antibodies to the cell surface are known in the art, and may include, for example, coupling using glutaraldehyde- or maleimide-activated antibodies. Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Biotinylation can be accomplished by, for example, treating the antibodies with dithiothreitol followed by the addition of biotin maleimide.

Antibodies are contacted with a bodily fluid from a subject at least for a time sufficient for the antibody to bind to a marker used to diagnose a heart condition. For example, an antibody may be contacted with a bodily fluid for at least about 10 minutes, 30 minutes, 1 hour, 3 hours, 5 hours, 7 hours, 10 hours, 15 hours, or 1 day.

The level of urotensin and additional markers of a heart condition may be detected and/or quantitated using an immunoassay. Immunoassays may be competitive or non-competitive. Such assays, both homogeneous and heterogeneous, are well-known in the art, wherein the analyte to be detected is caused to bind with a specific binding partner, such as an antibody, which has been labeled with a detectable species, such as a latex or gold particle, a fluorescent moiety, a biotinylated moiety, an enzyme, an electrochemically active species, etc. Alternatively, the analyte could be labeled with any of the above species and competed with limiting amounts of specific antibody. The presence or amount of analyte present is then determined by detection of the presence or concentration of the label. Such assays may be carried out in the conventional way using a laboratory analyzer or with point of care or home testing device, such as the lateral flow immunoassay as described in EP291194.

In certain embodiments, an immunoassay is performed by contacting a sample from a subject to be tested with an appropriate antibody under conditions that facilitate immunospecific binding between the antibody and marker(s) if present, and detecting and/or quantitating the amount of immunospecific binding by the antibody. In the context of the disclosed invention, “immunospecific” means that the antibody will bind specifically to urotensin. Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays.

For example, a marker can be detected in a bodily fluid by means of a two-step sandwich assay. In the first step, a capture reagent (e.g., an anti-marker antibody) is used to capture the marker. The capture reagent can optionally be immobilized on a solid phase. In the second step, a directly- or indirectly-labeled detection reagent is used to detect the captured marker. In one embodiment, the detection reagent is an antibody. In another embodiment, the detection reagent is a lectin. Any lectin can be used for this purpose that preferentially binds to the marker rather than to other proteins that share the antigenic determinant recognized by the antibody. In a preferred embodiment, the chosen lectin binds to the marker with at least 2-fold, 5-fold or 10-fold greater affinity than to other proteins that share the antigenic determinant recognized by the antibody. A lectin that is suitable for detecting a given marker can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174.

In other embodiments, a lateral flow immunoassay device may be used in the “sandwich” format wherein the presence of sufficient marker in a bodily fluid will cause the formation of a “sandwich” interaction at the capture zone in the lateral flow assay. The capture zone as used herein may contain capture reagents such as antibody molecules, antigens, nucleic acids, lectins, and enzymes suitable for capturing urotensin and other markers described herein. The device may also incorporate one or more luminescent labels suitable for capture in the capture zone, the extent of capture being determined by the presence of analyte. Suitable labels include fluorescent labels immobilized in polysterene microspheres. Microspheres may be coated with immunoglobulins to allow capture in the capture zone.

Other assays that may be used in the methods of the invention include, but are not limited to, flow-through devices. In a flow-through assay, one reagent (usually an antibody) is immobilized to a defined area on a membrane surface. This membrane is then overlaid on an absorbent layer that acts as a reservoir to pump sample volume through the device. Following immobilization, the remainder of the protein-binding sites on the membrane are blocked to minimize non-specific interactions. When the assay is used, a bodily fluid sample is added to the membrane and filters through the matrix, allowing any marker specific to the antibody in the sample to bind to the immobilized antibody. In an optional second step (in embodiments wherein the first reactant is an antibody), a tagged secondary antibody (an enzyme conjugate, an antibody coupled to a colored latex particle, or an antibody incorporated into a colored colloid) may be added or released that reacts with captured marker to complete the sandwich. Alternatively, the secondary antibody can be mixed with the sample and added in a single step. If a marker is present, a colored spot develops on the surface of the membrane.

In another embodiment, urotensin may be used as a diagnostic marker to determine the stage or severity of a heart condition in a subject. Urotensin may be quantitated in combination with a second marker indicative of a heart condition in a bodily fluid by use of an immunoassay. A diagnosis may be made based upon the results obtained from a healthy individual or individuals.

In an additional embodiment, urotensin may be used to identify subjects at risk for developing a heart condition. In this method, subjects with identified risk to develop a heart condition may be monitored for changes in urotensin levels quantitated from bodily fluid by an immunoassay.

In another embodiment, detection of increased urotensin levels during an acute myocardial infarction may be used to identify patients who are candidates for coronary revascularization therapies. Coronary revascularization is the process of restoring the flow of oxygen and nutrients (blood) to the heart. Coronary revascularization therapies include, but are not limited to, thrombolytic therapies (including, for example, treatment with anistreplase, streptokinase, urokinase, APSAC (acetylated plasminogen streptokinase activator complexes), tissue plasminogen activator (Alteplase, Reteplase, and Tenecteplase) and single chain urokinase plasminogen activator), adjunctive and conjunctive therapies (including, for example, treatment with heparin, aspirin, antagonists to platelet glycoprotein IIb/IIIa receptor, beta-blockers, angiotensin-converting enzyme inhibitors, and lipid-lowering agents), and mechanical revascularization therapies (including, for example, coronary artery bypass surgery, angioplasty (balloon angioplasty performed alone or after atherectomy), angioplasty performed with or without stent implantation (stents may be coated with a drug that inhibits restenosis)).

In a further embodiment, urotensin may be used a marker to determine the extent of cardiac damage incurred during angioplasty. In this method, urotensin levels may be quantitated in a subject's bodily fluid prior to angioplasty to establish a base level and then monitored for an increase during and after the angioplasty procedure. An increase in urotensin levels may indicate that cardiac damage occurred during reperfusion.

In another embodiment, the invention provides a method for monitoring the effect of therapy administered to a subject having a heart condition associated with up-regulated urotensin levels. In this method, urotensin levels may be quantitated from a bodily fluid by an immunoassay prior to the commencement of therapy to establish a base level for the patient. During the course of treatment, urotensin levels will be monitored for deviations from this base level to indicate whether the therapy is effective.

4. Kits

The invention also provides a kit for quantitating of urotensin and other markers useful in detecting an increased risk of a heart condition. Said kit may comprise one or more reagents for quantitating the level of urotensin in a bodily fluid. The one or more reagents may comprise an antibody that is immunospecific for urotensin. The kit may further comprise one or more reagents (i.e. an antibody) for measuring the level of a second marker indicative of the same disease.

A kit of the invention may additionally comprise one or more of the following: (1) instructions for using the kit for determining the level of a protein or a signal peptide; (2) a labeled binding partner to any antibody present in the kit; (3) a solid phase (such as a reagent strip) upon which any such antibody is immobilized; and (4) a label or insert indicating regulatory approval for diagnostic, prognostic, or therapeutic use or any combination thereof. If a labeled binding partner to the antibody is not provided, the antibody itself can be labeled with a detectable marker, e.g., a chemiluminescent, enzymatic, fluorescent, or radioactive moiety.

### EXEMPLIFICATIONS

The invention, having been generally described, may be more readily understood by reference to the following examples, which are included merely for purposes of illustration of certain aspects and embodiments of the present invention, and are not intended to limit the invention in any way.

### Example 1

### Urotensin Levels in Heart Failure Patients

Study Populations

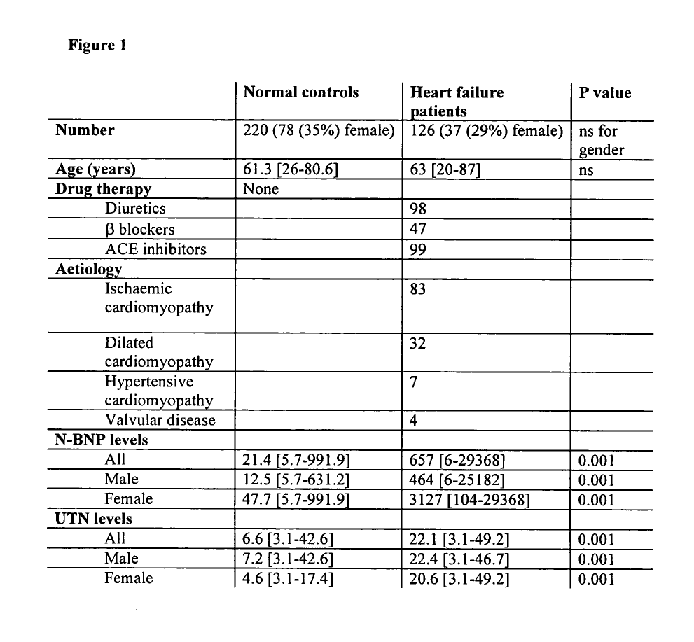

Patients with heart failure were recruited from the Leicester Royal Infirmary clinics and wards. All patients had a clinical diagnosis of heart failure (resulting from heart disease, as indicated in FIG. 1) and echocardiographically confirmed ejection fractions below 45%. Normal controls were age and gender matched with the heart failure patients, were on no medication, and had echocardiographically confirmed ejection fractions greater than 55%. Patient characteristics are reported in FIG. 1.

10 mls of blood was obtained from each patient by venipuncture after 15 min bed rest and mixed in ice-cold tubes containing EDTA and aprotinin. Plasma recovered following centrifugation was stored at −70° C. until assayed.

Assay of N-BNP

The assay for N-terminal proBNP (N-BNP) was based on the non-competitive N-BNP assay described by Karl et al. (Scand J Clin Lab Invest Suppl 1999;230:177-181). Rabbit polyclonal antibodies were raised to the N-terminal (amino acids 1-12) and C-terminal (amino acids 65-76) of the human N-BNP. IgG from the sera was purified on protein A sepharose columns. The C-terminal directed antibody (0.5 μg in 100 μL for each ELISA plate well) served as the capture antibody. The N-terminal antibody was affinity purified and biotinylated. Aliquots (20 μL) of samples or N-BNP standards were incubated in the C-terminal antibody coated wells with the biotinylated antibody for 24 hours at 4° C. Following washes, streptavidin labelled with methyl-acridinium ester (streptavidin-MAE, 5×10⁶ relative light units /ml) (Hart & Taaffe, J Immunol Methods 1987; 101: 91-96) was added to each well. Plates were read on a Dynatech MLX Luminometer as previously described (Hughes et al. Clin Sci 1999; 96: 373-380). The lower limit of detection was 5.7 fmol/ml of unextracted plasma. Within and between assay coefficients of variation were acceptable at 2.3 and 4.8%, respectively. There was no cross-reactivity with ANP, BNP or CNP.

Assay of Urotensin II

Antibody specific for the cyclic form of UTN was obtained from Phoenix Pharmaceuticals Inc., Belmont, Calif. Biotinylated UTN purified on reverse phase HPLC served as the tracer. A competitive assay using C₁₈ extracts of plasma was utilized, incubating 50 ng of the antibody with extracts or standards (ranging from 1 to 2000 fmol per well) in 100 μl of assay buffer (as described in 12). After 24 h of incubation at 4° C., the biotinylated UTN tracer was added (250 fmols per well). Immunoprecipitates were recovered in ELISA plates coated with anti-rabbit IgG (100 ng/well). Following washes and incubation with streptavidin-MAE, chemiluminescence was elicited as described above. Intra- and interassay coefficients of variation were 2.3 and 8.1%, respectively, with no cross-reactivity for BNP or N-BNP. The lower limit of detection was 3.1 fmol/ml.

Statistical analyses were performed on SPSS Version 11. Data are presented as medians (ranges). Comparisons were by Kruskal-Wallis analysis of variance and receiver operating characteristic (ROC) curves were plotted. Correlation analysis employed Spearman's rho (rₛ). A value of P<0.05 was considered statistically significant.

Results & Discussion

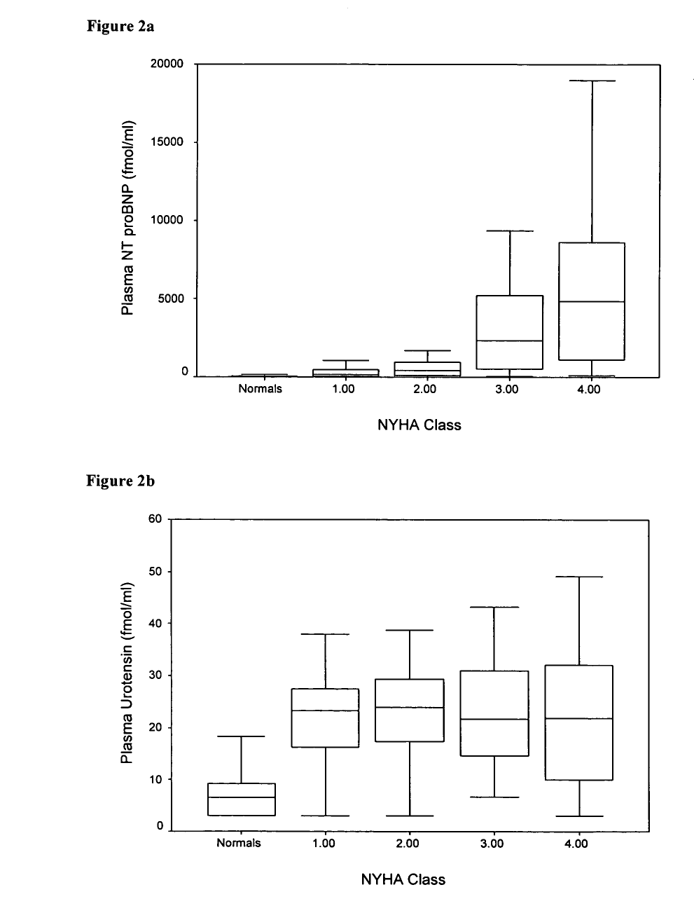

The results from this experiment are shown in FIG. 1 which illustrates the characteristics of the normal and heart failure patients, who were matched for age and gender. As expected, N-BNP was significantly elevated in heart failure patients. In the normal population, there was a positive correlation of N-BNP with increasing age (rₛ=0.41, P<0.001) and females had higher levels than males (P<0.001, FIG. 1). N-BNP increased with increasing NYHA class (FIG. 2a, P<0.001 by Kruskal Wallis test). Plasma UTN was also elevated in heart failure patients (FIG. 1), but there was no correlation with age. In contrast to N-BNP, levels were lower in females compared to males (P<0.001, FIG. 1). Plasma UTN was not affected by increasing NYHA class (FIG. 2b). Both N-BNP and UTN were elevated in heart failure patients irrespective of gender (P<0.001 for all comparisons). N-BNP and UTN were also modestly correlated (rₛ=0.35, P<0.001).

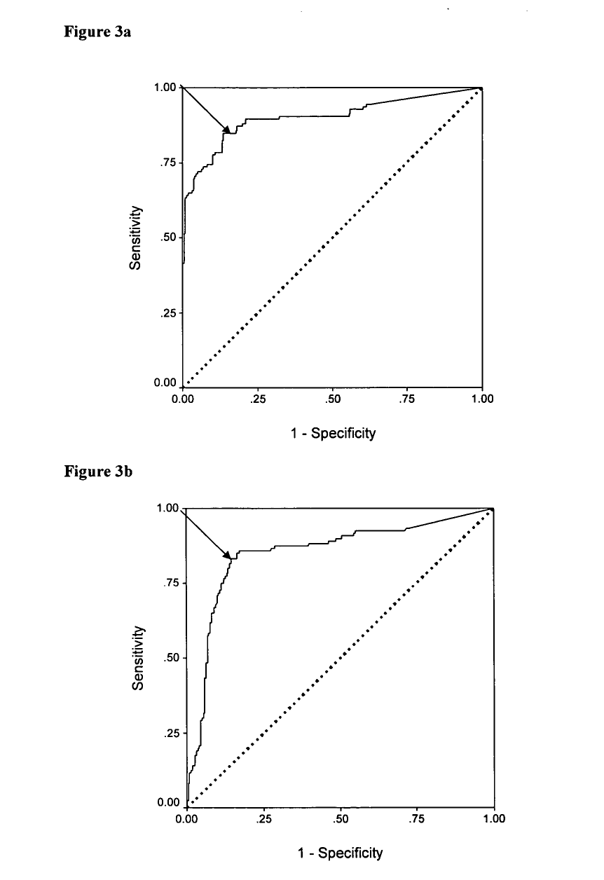

In the heart failure patients, plasma UTN levels were not dependent on use of diuretics, beta blockers or ACE inhibitors. As shown in FIG. 3, ROC curves for the detection of heart failure for both peptides revealed areas of 0.90 for N-BNP (FIG. 3a) and 0.86 for UTN (FIG. 3b) (P<0.001 compared to the diagonal reference line). The arrows indicate the distance between the top left comer and the curve; a shorter distance between the top left comer and the curve indicates a test with higher sensitivity and specificity.

Using the univariate general linear model procedure on SPSS, and entering age as a covariate and gender and NYHA class as factors, analysis of the log normalized N-BNP levels in the heart failure patients yielded an r² of 0.446 for the model (P<0.001) with age, gender, and NYHA class as significant predictive variables (P<0.034, 0.002 and 0.001 respectively). None of these factors were identified as predictive variables of UTN (r²=0.058). Thus, UTN levels in heart failure patients are elevated irrespective of age, gender or NYHA class.

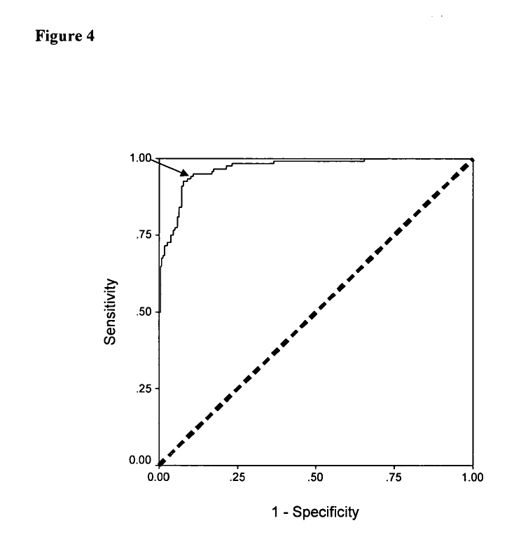

From the comparison of the graphs in FIGS. 2, one may see that the measurement of plasma UTN in combination with that of plasma N-BNP is able to yield greater information concerning the diagnosis of heart failure than measurement of N-BNP alone. Namely, levels of N-BNP only start to become elevated upon progression to NYHA Class 3, whereas plasma UTN levels are shown to be elevated in patients with an NYHA class of greater than 1. Thus, measurement of these two markers may lead to early stage identification of heart disease. For example, combining N-BNP and UTN levels using logistic regression analysis yields a new prognostic index (the predicted probability) which has an even larger ROC area under the curve of 0.968 than either N-BNP or UTN alone (FIG. 4, indicated by the arrow), indicating an independent contribution from both measured analytes that has increased the specificity and sensitivity of diagnosis of heart failure.

### Example 2

### Urotensin Levels in ACS Patients

Study Populations

486 patients admitted to the Leicester Royal Infirmary with acute coronary syndromes were studied. Acute myocardial infarction (MI) was defined as presentation with at least two of three standard criteria, i.e. appropriate symptoms, acute ECG changes of infarction (ST elevation, new LBBB), and a rise in creatine kinase (CK) to at least twice the upper limit of normal, i.e. >400 IU/L. Unstable angina (UA) patients were defined as having no acute ST elevation on their ECGs and CK was less than twice the upper limit of normal. 372 patients had MI and 114 had UA. These patients were compared with 130 normal controls of similar age and gender, with no previous cardiac history.

Endpoints in Myocardial Infarction and Unstable Angina Patients

End-points were defined as cardiovascular morbidity (rehospitalization with a further episode of myocardial infarction or unstable angina) following discharge from the index hospitalization.

Blood Sampling and Plasma Extraction

In all subjects, 20 ml of peripheral venous blood was drawn into pre-chilled Na-EDTA (1.5 mg/ml blood) tubes containing 500 IU/ml aprotinin after a period of 15 min bed rest. In MI and UA patients, a single blood sample was taken between 72-96 hours after symptom onset. After centrifugation at 3000 rpm at 4° C. for 15 min, plasma was separated and stored at −70 ° C. until assay. Prior to assay, plasma was extracted on C₁₈ Sep-Pak (Waters) columns and dried on a centrifugal evaporator.

Assay of UTN

The assay of UTN was carried out as previously described in Ng, et al., Circulation 2002, 106: 2877-2880. In brief, antibody specific for UTN was obtained from Peninsular Laboratories, CA. Biotinylated UTN purified on reverse phase HPLC served as the tracer. A competitive assay using C₁₈ extracts of plasma was utilized, incubating 50 ng of the antibody with extracts or standards in immunoassay buffer consisting of (in mmol/l) NaH₂PO₄1.5, Na₂HPO₄8, NaCl 140, EDTA 1 and (in g/l) bovine serum albumin 1, azide 0.1. After 24 h of incubation at 4° C., biotinylated UTN tracer was added (250 fmols/well). Immunoprecipitates were recovered in anti-rabbit IgG coated ELISA plates. Following washes and incubation with streptavidin-MAE, chemiluminescence was elicited as described in Ng, et al., Circulation 2002, 106: 2877-2880 and Ng, et al., Clinical Science 2002, 102: 411-416.

Assay BNP and N-BNP

Antibody specific for BNP was obtained from Peninsular Laboratories, CA. Biotinylated BNP purified on reverse phase HPLC served as the tracer. A competitive assay using C₁₈ extracts of plasma was utilized, incubating 25 ng of the antibody with extracts or standards in immunoassay buffer, as detailed under assay for UTN. The assay for N-BNP was a 2-site non-competitive assay, employing antibodies against the N- and C-terminal of human N-BNP, as described in Ng, et al., Circulation 2002, 106: 2877-2880 and Omland, et al., Circulation 2002, 106: 2913-8.

Statistical Analysis

Statistical analysis was performed using SPSS Version 11.0 (SPSS Inc, Chicago, Mich.). Data are presented as mean±SEM or median (range) for data with non-Gaussian distribution, which were log transformed prior to analysis. For continuous variables, one-way analysis of variance (ANOVA) was used and post-hoc comparisons sought with Bonferonni's test. The interaction of multiple independent variables was sought using the univariate General Linear Model procedure with least significant difference P values reported. Spearman correlation analysis was performed (rₛ are reported) and box plots were constructed consisting of medians, boxes representing interquartile ranges and the whiskers representing the 2.5^{th} to the 97.5^{th} percentile. Receiver operating characteristic curves (ROC) were constructed for the detection of ACS compared to normal subjects. P values below 0.05 were considered significant.

Results & Discussion

UTN in Plasma of Normal and ACS Patients

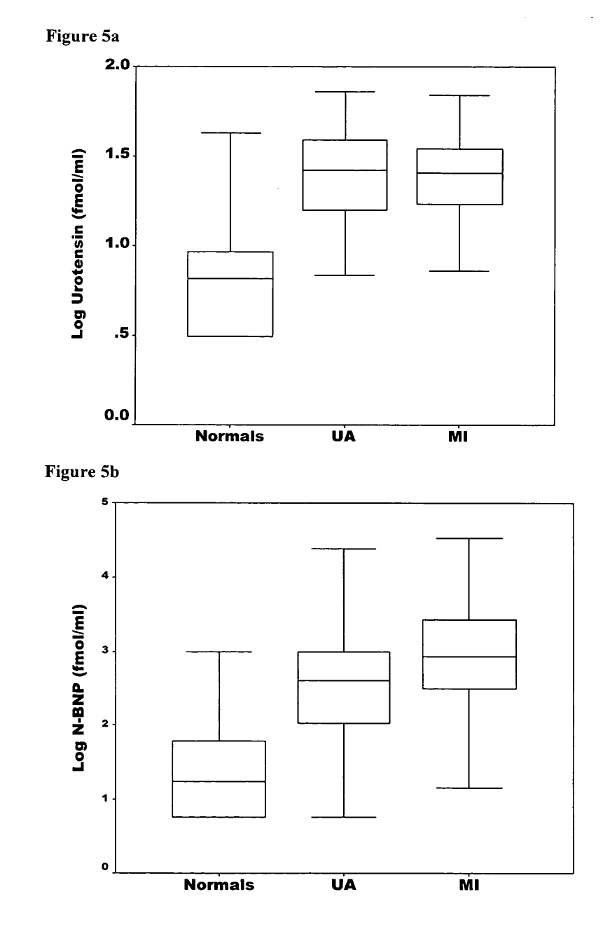

UTN was detectable in plasma extracts of almost all subjects. Some extracts were below the detection limit of our assay (<3.1 fmol/ml). The levels of UTN in plasma of ACS patients (both MI and UA) were significantly higher than that of normal controls (FIG. 5a, ANOVA P<0.0005). Bonferonni's test confirmed UTN levels in UA (P<0.0005) or MI (P<0.0005) were significantly higher than normal controls. The levels of N-BNP in plasma of ACS patients (both MI and UA) were also significantly higher than that of normal controls (FIG. 5b, ANOVA P<0.0005). Bonferonni's test confirmed N-BNP levels in UA (P<0.0005) or MI (P<0.0005) were significantly higher than normal controls. In addition, N-BNP levels in MI were higher than that in UA (P<0.0005). Levels of UTN and N-BNP were correlated (rₛ=0.354, P<0.0005).

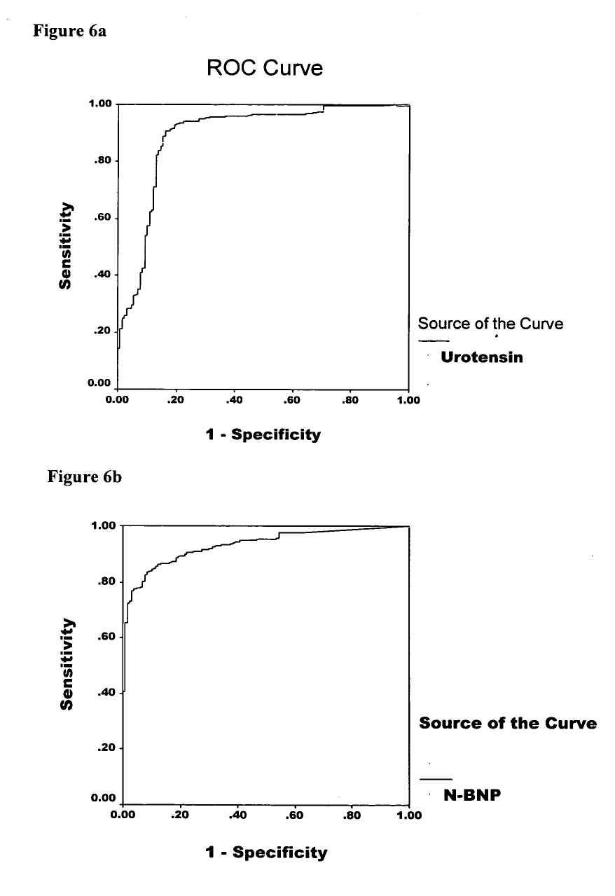

FIG. 6a illustrates a receiver operating characteristic curve for the diagnosis of ACS (MI and UA) using plasma UTN levels. The ROC area under the curve (ROC AUC) was 0.89 for UTN, significantly different (P<0.0005) from the diagonal (AUC of 0.50), but similar to that of N-BNP (ROC AUC 0.93, FIG. 6b). Both markers thus have utility in the identification of patients with ACS. At a level of UTN of 9.1 fmol/ml, there was a 94% sensitivity, 74% specificity for the diagnosis of ACS, with positive predictive values of 93% and a negative predictive value of 77%. These figures allow effective ruling-in of the diagnosis of ACS. Appropriately changing the cut-off values can also improve the utility of UTN to rule out an ACS event.

Plasma UTN and Prognosis of ACS Events

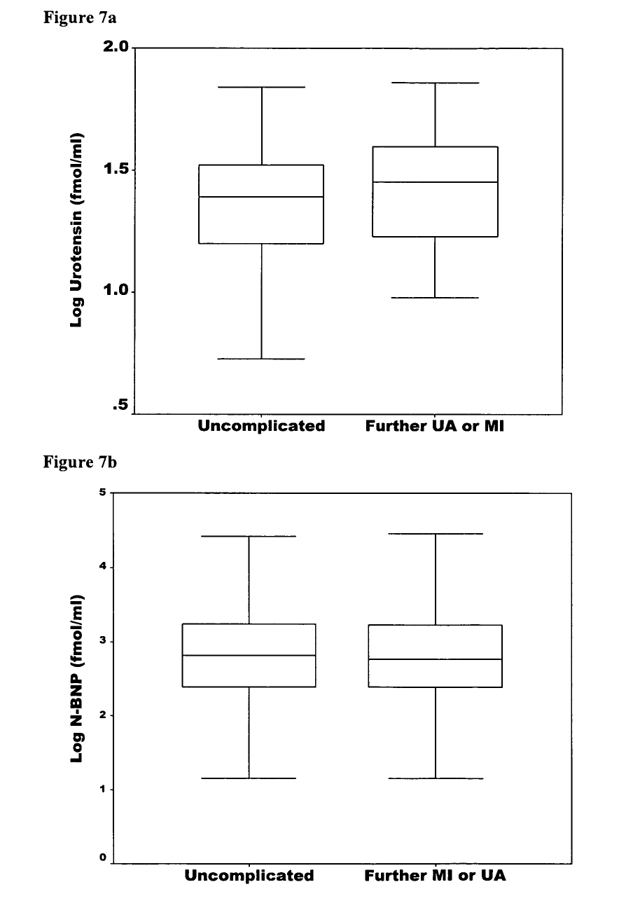

FIG. 7a shows that levels of UTN in patients who had an index admission with ACS, but were subsequently either readmitted with MI or UA, or remained well and were not rehospitalized. Data was available on 447 patients. It can be seen UTN is elevated in the 98 patients who readmitted with MI or UA (P<0.005), compared to the 349 patients who had an uncomplicated clinical course. In contrast, plasma N-BNP was not different in patients who had an uncomplicated course, compared to those subsequently rehospitalized with MI or UA (FIG. 7b). UTN can be an effective marker for future ACS events in patients admitted to hospital with ACS, and this can assist in the risk stratification and planning of therapeutic strategies for such higher risk patients.

### Example 3

### Urotensin Levels in Stable Angina Patients Undergoing Coronary Angiography

Study Population and Blood Sampling

Blood samples were obtained from 23 patients with stable angina undergoing coronary angiography. 13 of these patients had balloon angioplasty in addition to angiography. Blood samples were taken before angiography (basal), 2 hours, 6 hours and 24 hours after angiography (or angioplasty). Samples were extracted on C₁₈ columns and assayed for UTN and BNP as described above.

Results & Discussion

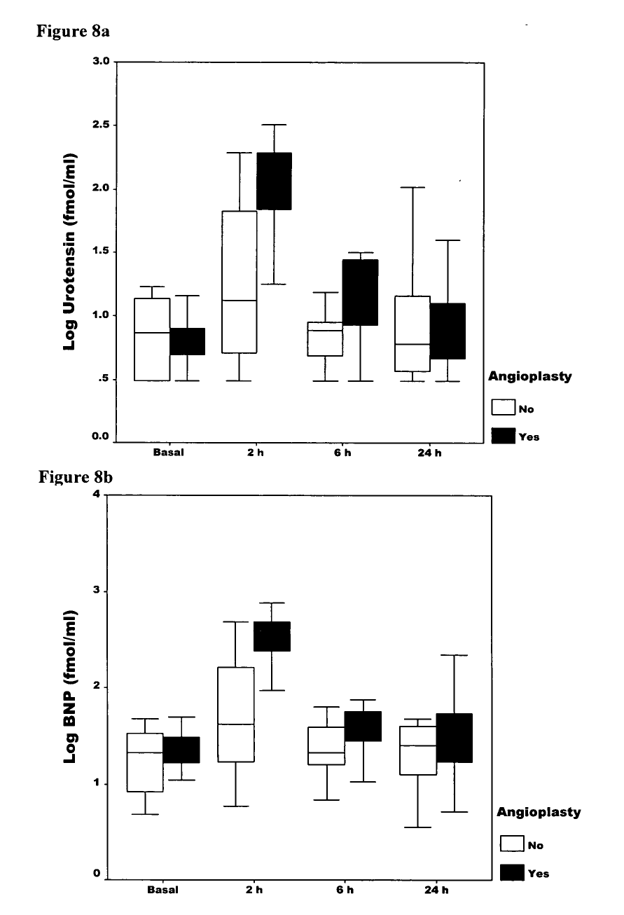

FIG. 8a shows the plasma levels of UTN during angiography with or without angioplasty. Levels change significantly with time (P<0.0005), peaked at 2 hours after the procedure (P<0.0005 compared to all other time points), and differed between those with angioplasty compared to those without angioplasty (P<0.011).

The levels of BNP also changed significantly with time (P<0.0005), peaked at 2 hours after the procedure (P<0.0005 compared to all other time points), and also differed between those with angioplasty compared to those without angioplasty (P<0.008) (FIG. 8b).

Although high levels of both markers are evident at 2 hours after the angioplasty, higher levels may be evident before 2 hours or between 2 and 6 hours. The optimal time for obtaining the blood sample may be in the range of 10 min to 6 hours after the procedure.

BNP is an established marker of cardiac damage, being released during cardiac ischaemia. However, the acute secretion of UTN during cardiac ischaemia (e.g. during balloon angioplasty) is unexpected. Cardiac ischaemia can be transient and mild during this procedure, but the median 10 fold rise in UTN suggests it could be a very sensitive indicator of cardiac ischaemia and may provide information on the degree of cardiac damage incurred during angioplasty. This should complement information obtained from other peptide assays, e.g. troponins and BNP.

In addition, the acute response of UTN to balloon angioplasty suggests it may be a marker for the early diagnosis of an acute coronary syndrome, since levels peak within 2 hours. Current markers of myocardial ischaemia which are released rapidly from the myocardium during an ACS event (such as myoglobin) may be non-specific for cardiac muscle, and additional markers (such as UTN and BNP) can increase the specificity of myoglobin.

Equivalents

The invention provides in part methods of diagnosing a heart condition in a subject by quantitating urotensin levels in the subject's bodily fluid. While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

All publications and patents mentioned herein are hereby incorporated by reference in their entireties as if each individual publication or patent was specifically and individually indicated to be incorporated by reference. In case of conflict, the present application, including any definitions herein, will control.

Also incorporated by reference are: UK Patent Application No. 0216191.7, UK Patent Application No. 0216500.9, UK Patent Application No. 0216505.8, UK Patent Application No. 0401085.6, and U.S. patent application Ser. No. 10/618,567.

1. A method for diagnosing a heart condition in a subject by quantitating the level of urotensin present in the subject's bodily fluid, wherein a level greater than 10 fmol/ml indicates that the subject has or is at risk of developing a heart condition.

2. The method of claim 1, wherein the heart condition is an acute coronary syndrome (ACS).

3. The method of claim 1, wherein the level of urotensin is quantitated using an immunoassay.

4. The method of claim 3, wherein the immunoassay is a lateral flow format.

5. The method of claim 1, wherein the subject is human.

6. The method of claim 1, wherein the bodily fluid is selected from the group consisting of plasma, whole blood, serum, interstitial fluid, urine, and saliva.

7. The method of claim 1, further comprising quantitating a second marker in a subject's bodily fluid, wherein a level of the second marker that is greater than the normal level indicates that the subject has or is at risk of developing a heart condition.

8. The method of claim 7, wherein the second marker is selected from the group consisting of brain natriuretic peptide, atrial natriuretic peptide, C-type natriuretic peptide, troponin, creatine kinase MB isoform, and myoglobin.

9. A method for monitoring cardiac damage incurred by a subject as a result of a procedure comprising quantitating the level of urotensin in the subject's bodily fluid prior to the procedure and comparing the amount to the level of urotensin in the subject's bodily fluid during and after the procedure, wherein a higher amount after the procedure than the amount quantitated prior to the procedure indicates that cardiac damage occurred in the subject as a result of the procedure.

10. The method of claim 9, wherein the procedure is angioplasty.

11. The method of claim 9, wherein the quantitating step is performed using an immunoassay.

12. The method of claim 11, wherein the immunoassay is a lateral flow format.

13. The method of claim 9, wherein the bodily fluid is selected from the group consisting of plasma, whole blood, serum, interstitial fluid, urine, and saliva.

14. The method of claim 9, further comprising measuring the level of a second marker selected from the group consisting of brain natriuretic peptide, atrial natriuretic peptide, C-type natriuretic peptide, troponin, creatine kinase MB isoform, and myoglobin.

15. A method of monitoring the treatment of a subject with acute coronary syndrome (ACS) comprising: (a) quantitating the urotensin level in the subject's bodily fluid; (b) comparing the urotensin level to a normal level; and (c) administering a coronary revascularization therapy to the subject when the subject is determined to have an increased level of urotensin.

16. The method of claim 15, wherein the quantitating step is performed using an immunoassay.

17. The method of claim 16, wherein the immunoassay is a lateral flow format.

18. The method of claim 15, wherein the bodily fluid is selected from the group consisting of plasma, whole blood, serum, interstitial fluid, urine, and saliva.

19. The method of claim 15, further comprising measuring the level of a second marker selected from the group consisting of brain natriuretic peptide, atrial natriuretic peptide, C-type natriuretic peptide, troponin, creatine kinase MB isoform, and myoglobin.

20. A kit for diagnosing a heart condition in a subject comprising at least one reagent for quantitating urotensin in the subject's bodily fluid and instructions for use.

21. The kit of claim 20, wherein the reagent for quantitating urotensin is an antibody.

22. The kit of claim 20, further comprising a reagent that binds to a second marker of a heart condition.

23. The kit of claim 22, wherein the second marker is selected from the group consisting of brain natriuretic peptide, atrial natriuretic peptide, C-type natriuretic peptide, troponin, creatine kinase MB isoform, and myoglobin.
